(19) United States
(12) Patent Application Publication
(10) Pub. No.: US 2015/0313537 A1
(43) Pub. Date: Nov. 5, 2015

(54) BIOELECTRONIC SIGNAL PROCESSOR AND PROCEDURE TO LOCATE AND DESTROY PANCREATIC CANCER CELLS

(71) Applicant: Neuro Code Tech Holdings, LLC, Albuquerque, NM (US)

(72) Inventor: Eleanor L. Schuler, Rio Rancho, NM (US)

(73) Assignee: Neuro Code Tech Holdings, LLC

(21) Appl. No.: 14/694,019

(22) Filed: Apr. 23, 2015

Related U.S. Application Data
(60) Provisional application No. 61/986,988, filed on May 1, 2014.

(51) Int. Cl. A61B 5/00; A61N 5/06; A61N 1/05

(52) U.S. Cl. CPC A61B 5/4836; A61B 5/0084; A61N 1/05; A61N 5/0601

(57) ABSTRACT

Methods and systems for treating pancreatic cancer. A signal associated with pancreatic cancer in a body can be detected and recorded, and then reprogrammed to generate a confounding treatment signal with respect to the pancreatic cancer. The confounding treatment signal can be aimed at the pancreatic cancer in the body to shut down pancreatic cancer cells associated with said pancreatic cancer. The treatment signals can be provided as tiny encoded signals powered by ultra-low voltage approximated to the power level of the actual live pancreatic cancerous cell targets. An audio-light-optic system can seek out spreading pancreatic cancer cells that have moved onto nerves and blood vessels and the nearby organs, which include, for example, the liver, stomach, gall-bladder, spleen, and duodenum. Once the aberrant pancreatic cells are identified, the confounding treatment signals can be applied to destroy them.

### CROSS-REFERENCE TO PROVISIONAL APPLICATION

This application claims priority under 35 U.S.C. 119(e) to U.S. Provisional Patent Application Ser. No. 61/986,988, entitled “Bioelectronic Signal Processor and Procedure to Locate and Destroy Pancreatic Cancer Cells,” which was filed on May 1, 2014, the disclosure of which is incorporated herein by reference in its entirety.

### TECHNICAL FIELD

Embodiments generally relate to the field of bioelectronic medicine or “electroceuticals” and in particular to the treatment of cancer. Embodiments further relate to the alteration of cancer cell internal electrical signals so as to trigger and induce cell death. Embodiments additionally relate to inducing apoptosis (programmed cell death) in cancer cells due to reprogramming the intra-cellular operational communication network. Embodiments further relate to the treatment of pancreatic cancer.

### BACKGROUND OF THE INVENTION

Pancreatic cancer is the fourth leading cause of cancer death in the United States. It has been estimated that approximately 43,000 people per year are diagnosed with pancreatic cancer in the United States and that approximately 37,000 will die from the disease yearly. While the incidence rates have been declining for many cancers, the incidence rate for pancreatic cancer continues to increase. Pancreatic cancer is one of the few cancers for which the relative survival rate has not improved substantially in over 40 years. Pancreatic cancer has the lowest relative survival rate of all the cancers tracked by both the American Cancer Society and the National Cancer Institute. It is estimated that 94% of pancreatic cancer patients will die within five years of diagnosis and only 6% will survive more than five years. 74% of patients die within the first year of diagnosis.

The pancreas gives rise to several serious malignant and benign neoplasms. Cancer of the pancreas ranks fourth as a cause of death in both sexes in the United States.

The pancreas is a glandular organ that is both an endocrine gland and a digestive organ. It is located behind the base of the stomach and in front of the spinal column. It is surrounded by many important organs whereto it can spread a cancer that arises from the pancreas itself. Pancreatic cancer is the 4^{th} most common cause of cancer-related deaths in the United States and probably around 12^{th} in the world. Pancreatic cancer is a glandular tumor of which 95% of cases are classified as an adenocarinoma.

Pancreatic cancer has a very poor prognosis. Unfortunately, 80% of patients usually survive only 6 to 10 months. Some patients only seek medical care: they are already in terminal-condition and only have a few days or weeks before they succumb to the disease. Pancreatic cancer is a terrible disease, which took Steve Jobs, the brains and power behind Apple Computer and its famous cellular Smartphone. In addition, a number of wealthy and famous people have been struck by pancreatic cancer, such as, for example, Richard Crenna, Joan Crawford, Sir Rex Harrison, Donna Reed, Lilly Pons, Juliet Prowse and Patrick Swayze, among many others. Former U.S. President Jimmy Carter lost his two sisters to pancreatic cancer.

Because the medical signs of pancreatic cancer are similar to many other diseases, there are some clues that should prompt a check-up by a doctor, especially if you have a genetic predisposition to cancers of the digestive tract or have any blood-relative that had pancreatic cancer. Here are some possible signs of pancreatic cancer:

1. Pain in the stomach that radiates into your back.

2. Jaundice noticeable by yellowing of the skin and the whites of your eyes.

3. Light or pale colored stools and/or dark urine.

4. Feeling tired and loss of appetite.

5. Unexplained weight loss.

6. Diabetes Mellitus or high blood sugar levels.

People with cancers that are completely operable have the best chance of survival. Unfortunately, only 10 to 20 out of 100 that are diagnosed with pancreatic cancer are still suitable for surgery.

As of 2010, pancreatic cancer resulted in 310,000 deaths globally. In the USA during 2010, there were 43,000 cases of pancreatic cancer and almost 37,000 of them died. The so-called “Carcinoma of the pancreas” has one of the highest fatality rates of all cancers. It is surpassed only by lung, colon, and female breast cancer concerning numbers of humans that eventually die from these particular malignancies. The incidence of pancreatic cancer stands more frightening due to the helplessness of the patient in knowing that the occurrence of pancreatic cancer is increasing. Pancreatic cancer is one of the few cancers for which the survival rate has not improved substantially in over the past 40 years.

The National Cancer Institute reports that 94% of pancreatic cancer patients will die within 5 years of diagnosis and only 6% will survive more than 5 years. Unfortunately, 74% of patients die within the first year of diagnosis.

Humans are composed of 100 trillion cells made up of more than 220 types or kinds of cells that have different duties or functions. Most people are unaware that all human cells have an electrical process that allows the operation of all the internal elements of each cell's life and allows the cell to contribute its operational duties to the human body it lives in. Cells can produce chemicals like tears, adrenalin, or bile for digestion, can be part of the muscles that allow the body to move about or operate vital organs such as the heart which electrically contracts the heart and circulates the blood supply. Normal cells can replace themselves when they wear out. Examples include replacing the lining of the digestive tract every three days and supplying new red blood cells every 90 days. Injured skin can replace it self in about a week in young people, longer in the aged. Normal cells usually cannot reproduce themselves more than about 70 times during the life-time of the human body.

However, the body can generate brand new cells when needed. Each of the normal cells strive to keep themselves healthy so as to perform appropriately.

One of the features that render cancer cells so lethal is that they can reproduce themselves continuously and establish a significantly large tumor. The cancerous tumor has no particular job except to reproduce more cancer, which it can do in as quickly as 10 to 24 hours.

A normal cell must keep itself healthy and perform its assigned duties, while cancer cells only live to reproduce more cancer continuously since it has no job to contribute to anything the body needs. The cancer is using its time by building a large tumor and to live for itself by taking from the body what it needs. When it has grown so large that it does no have enough oxygen to keep itself alive, it merely use an electrical signal to tell an artery to build a new bud on its artery and construct a new branch that leads itself right into the tumor so it can supply more oxygen for its continuous reproduction of large number of malignant cells. As the tumor grows, it keeps sending messages to the adjacent cancer cells only, as it does not communicate with normal cells, which may be right next to it. Tumors can grow to a large size, given the necessary time, and can be composed of millions of cells.

Cells are very tiny. A pencil dot or ballpoint pen dot could represent 40 to 400 cells. All cells have an electrical process to operate its own activity and to contribute something that the human body needs. It is only the cancer structure that is selfish just to keep on growing which can crowd out and strangle normal cells. As the cancer grows, it can disrupt the production of endocrine or exocrine secretions that the body needs for digestion or energy. A cancer tumor does nothing good for the body it ultimately kills, given enough reproductive time.

It is well known that cancer cells have escaped the control mechanisms that regulate growth. They have a ravenous appetite for glucose and have chromosomal imbalances involving networks of damaged or aberrant genes as part of the cancerization process.

Cancer cells exhibit altered electrochemical properties and variant distribution of their electrical signals as compared to their healthy signals and tissues. Cancer cell walls move neutrinos differently than normal cells and use their outer glycocalyx as a protective barrier to discourage attack by the body's immune system.

The glycocalyx is a carbohydrate-enriched very thin plasma membrane cell encasement. The glycocalyx carries a strong negative electrical charge. This provides an effective protective shield to repel the body's immune system. The immune system elements are also negatively electrically charged. Negative against negative repels, so the immune system is unable to attack the malignant cells of a tumor. This is why we must use the scientific cell electric signal processor to alter the entire cancer's electrical process so it cannot function.

Malignant cells have higher levels of sodium, which contributes to its electrochemical ability to generate internal communication signals for the cancer. Most likely both the plasma membrane charge and the internal electrical metabolic and reproductive signaling carry the instructions that drive the relentless spread of cancer clusters. The cancer cell features a well-established electrical constant, which is unlike healthy cells.

Therefore, a tactic that would alter the tissue electrical encodement of a cancer cluster is expected to interfere with the internal metabolic process and reproduction pace, as well as its resistance to an attack by encoded signals prepared to shut down the malignant cells, permanently.

The present inventor has been involved in the development of the premier bioelectronic technology of our time as outlined in a variety of bioelectronic medical treatment patents and patent applications covering the use of neuro-coded signaling technology. Validation of this bioelectronic technology is evidence by the fact that large pharmaceutical companies and organizations are now moving into the field of bioelectronics, albeit many years after the present inventor's initial patent application filings and without much in the way of intellectual property. For example, the monolithic international pharmaceutical giant GSK (GlaxoSmithKline) announced in 2013 that it was pursuing an effort toward the development of “electroceutical” or bioelectronic medicine (see “A Jumpstart-Start for Electroceuticals, Nature”, 11 Apr. 2013, Vol 496, pp. 159-161. Famm et al). Ironically, the present inventor's own research and thinking in the bioelectronic or ‘electroceuticals’ area was captured in patent filings many years prior to GSKs 2013 initiative.

To date, the primary approach to treating cancer based on bioelectronic technology has been outlined in patents and patent application publications by the present inventor. Such in inventions are disclosed in, for example, U.S. Patent Application Publication No. 2010/0286689 entitled “Method and System for Processing Cancer Cell Electrical Signals for Medical Therapy,” which published on Nov. 11, 2010; U.S. Patent Application Publication No. 2011/0270248 entitled “System and Method to Elicit Apoptosis in Malignant Tumor Cells for Medical Treatment,” which published on Nov. 3, 2011; U.S. Patent Application Publication No. 2011/0130754 entitled “Hybrid Scientific Computer System for Processing Cancer Cell Signals as Medical Therapy,” which published on Jun. 2, 2011; and U.S. patent application Ser. No. 12/334,212 entitled “Method to Switch-Off Cancer Cell Electrical Communication Codes as Medical Therapy,” which was filed on Dec. 12, 2008. U.S. Patent Application Publication Nos. 2010/0286689; 2011/0270248; and 2011/0130754; and U.S. patent application Ser. No. 12/334,212 are incorporated herein by reference in their entireties. Additionally, U.S. patent application Ser. No. 14/621,659 entitled “Encoded Bioelectronic Method and System and Calcium Treatment for Slaying Cancer by Rapid Triggering of Cellular Apoptosis and Karyorrhexis,” which was Feb. 13, 2015 is incorporated herein by reference in its entirety. U.S. patent application Ser. No. 14/656,142 entitled “Rapid Destruction of Malignant Tumors by Excitotoxicity and Osmotic-Shock Medical Tactics” which was filed on Mar. 12, 2015 is also incorporated herein by reference in its entirety.

### BRIEF SUMMARY

The following summary is provided to facilitate an understanding of some of the innovative features unique to the disclosed embodiments and is not intended to be a full description. A full appreciation of the various aspects of the embodiments disclosed herein can be gained by taking the entire specification, claims, drawings, and abstract as a whole.

It is, therefore, one aspect of the disclosed embodiments to provide for an improved cancer treatment technology.

It is another aspect of the disclosed embodiments to provide for a cancer specific analog/digital scientific computer system, which can be utilized to destroy pancreatic cancer tumors and their areas of metastatic spread.

It is another aspect of the disclosed embodiments to provide for a method and/or procedure for locating, targeting, and killing pancreatic cancer cells.

The aforementioned aspects and other objectives and advantages can now be achieved as described herein. A cancer specific analog/digital scientific computer system can be utilized to destroy pancreatic cancer tumors and their areas of metastatic spread. The system records and maps the intrinsic pancreatic cancer tumor's signals and sounds that control the malignancy, including its mitotic reproductive process. Cancer cells reproduce continuously during their lifetime whereby each nucleus divides and becomes two new cells in a continuous mitosis reproductive process, without end.

The bioelectronic system features both analog and digital components, and encodes treatment signals that are formed from the actual cancer signals which are reprogrammed into at least two confounding treatment-signals, which are prepared for transmission into the pancreatic cancer cells to shut-down the pancreatic cancer, permanently.

The treatment signals are formatted into confounding signals, which when transmitted from electrodes of the bioelectronic system into the tumor and any reachable metastatic colonies are designed to trigger necrosis, apoptosis, or karyorrhexis of the cancer cells that receive the treatment signals.

A diagnostic probe locates and identifies spurious or aberrant cancer cells that have spread to nearby organs, nerves, and blood vessels. Such a probe can utilize lights that heat cancer cell walls in such a way as to causes them to vibrate and make a sound that is different from the song of normal cells. An audio system coupled to the analog-digital bio-electronic computer detects the presence of small groups of cancerous cells and records their location.

### BRIEF DESCRIPTION OF THE DRAWINGS

The accompanying figures, in which like reference numerals refer to identical or functionally-similar elements throughout the separate views and which are incorporated in and form a part of the specification, further illustrate the embodiments and, together with the detailed description, serve to explain the principles of the present invention.

FIG. 1 illustrates a schematic diagram of a pancreas;

FIG. 2 illustrates a block diagram of bioelectronic medical system for locating and destroying pancreatic cancer cells, in accordance with a preferred embodiment;

FIG. 3 illustrates a diagram of a human body, particularly a torso and abdominal area, with possible points of entry thereon for locating and destroying pancreatic cancer cells in accordance with an alternative embodiment;

FIG. 4 illustrates a diagram of an example surgical operating room in which the disclosed procedure for locating and destroying pancreatic cancer cells may be carried out, in accordance with an alternative embodiment;

FIG. 5 illustrates a side view of a patient in preparation for locating and destroying pancreatic cancer cells therein, in accordance with an alternative embodiment;

FIG. 6 illustrates potential medical components and devices that can be utilized in accordance with the disclosed system and procedure for locating and destroying pancreatic cancer cells in accordance with an alternative embodiment; and

FIG. 7 is an example of a dated analog type computer or computing device that can be adapted for use or designed with contemporary electrical components for use with the disclosed embodiments herein.

### DETAILED DESCRIPTION

The particular values and configurations discussed in these non-limiting examples can be varied and are cited merely to illustrate at least one embodiment and are not intended to limit the scope thereof.

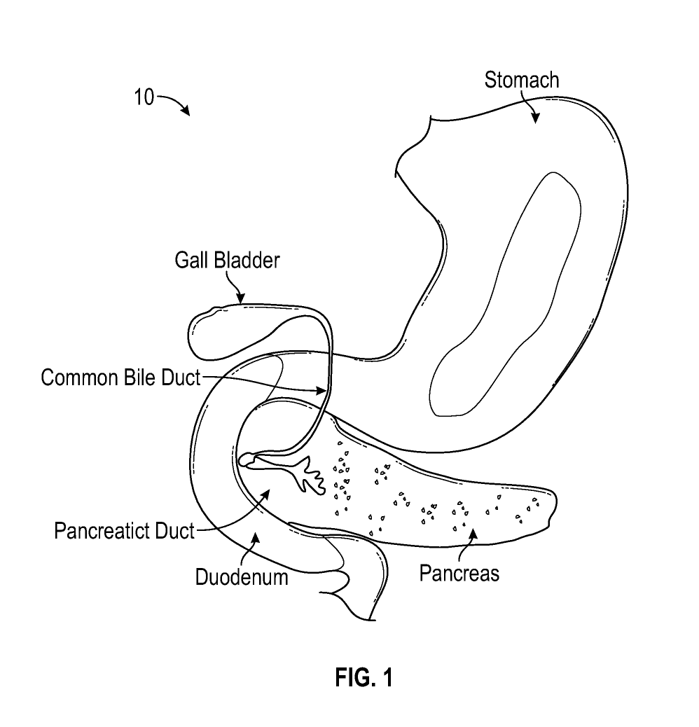

FIG. 1 illustrates a schematic diagram 10 of a pancreas including its location relative to the stomach, gall bladder, common bile duct, duodenum, and pancreatic duct. One of the reasons that pancreatic cancer is the most dangerous malignancy is that it is difficult to make an early diagnosis as the malignancy is reproducing and spreading to the close organs, blood vessels, and nerves found in the abdominal area that surrounds the pancreas.

The disclosed embodiments therefore focus on shutting-down cancer cell electrical operating systems and disturbing the tumorous structure command and communication systems that interconnect the individual cells that constitute the tumor. Each separate metastatic island of cancer cells that can be detected on the pancreas and the nearby organs and tissues including associated blood vessels or nerves will receive the appropriate dose of the confounding encoded electrical treatment signal.

A hybrid bioelectronic treatment system can locate and shut off the cellular internal operating electrical system of the cancer and take-down all possible cancer cell activity, which the treatment signals can make contact with. One such example of a hybrid bioelectronic treatment system that can be adapted for use with one or more embodiments is disclosed in U.S. Patent Application Publication No. 2011/0130754 entitled “Hybrid Scientific Computer System for Processing Cancer Cell Signals as Medical Therapy.”

The disclosed embodiments can be utilized to scan and inspect all the nearby organs and blood vessels for the pancreatic cancer cells, which may have spread or have traveled regionally, metastatically.

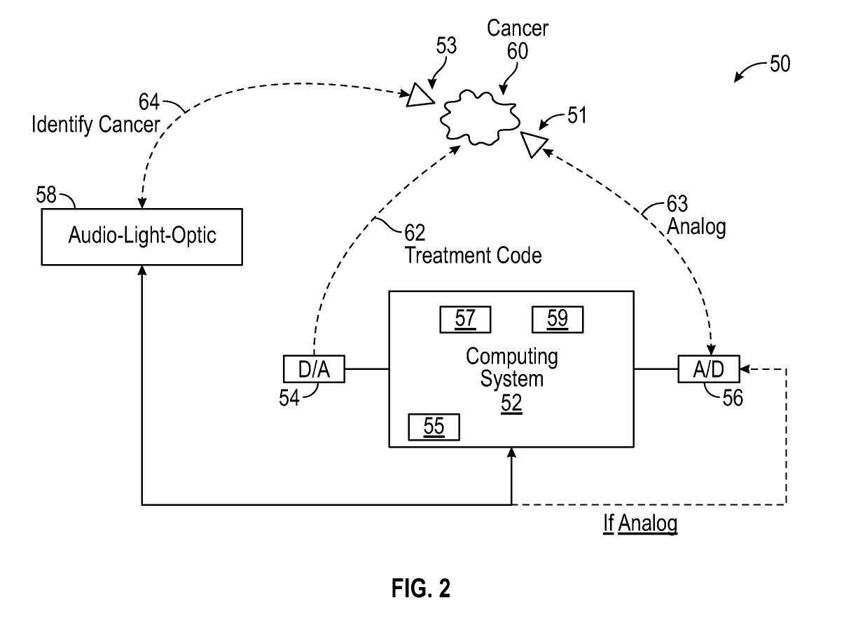

FIG. 2 illustrates a block diagram of bioelectronic medical system 50 for locating and destroying pancreatic cancer cells, in accordance with a preferred embodiment. One element of bioelectronic treatment system 50 shown in FIG. 2 features the use of a detector or illuminated-audio-detection system (i.e., audio-light-optic system) 58 to locate living cancer cells that have spread to the surface of the stomach, liver, gall bladder, duodenum, spleen, and of course, the pancreas, and other tissue. System 50 generally includes a computing system 52 which may include a signal processor 55. The system 50 can communicate electronically with a D/A (Digital to Analog) converter 54 and an ND (Analog to Digital) converter 56. Dashed arrow 62 shown in FIG. 2 illustrates cancer treatment codes delivered to a pancreatic cancer tumor or an area of suspected pancreatic cancer in a human body.

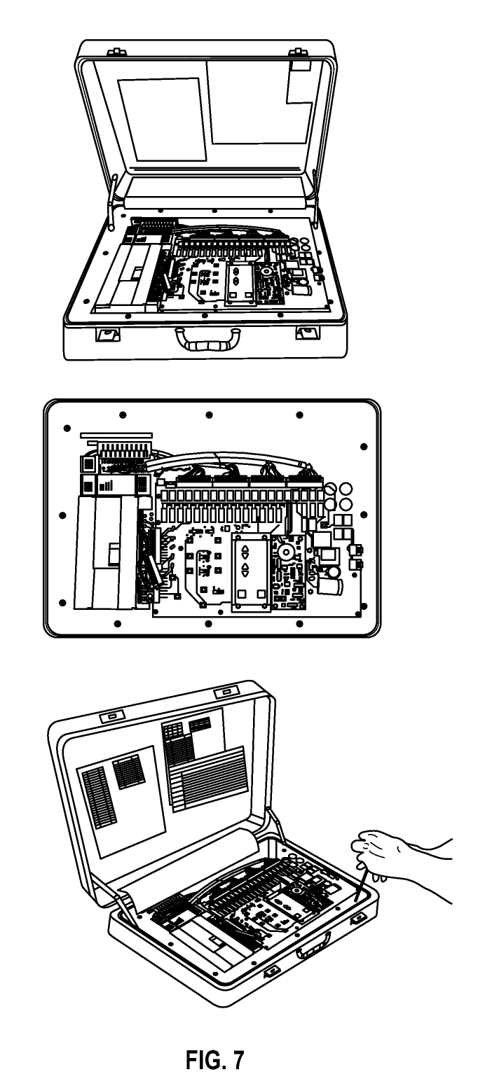

It can be appreciated that the computing system 52 may be implemented in the context of any number of data processing or computing systems or devices. For example, in one embodiment, computing system 52 may be a standalone desktop type computer. In another embodiment, computing system 52 may be a laptop computer or a hand held computing device such as, for example, a smartphone or table computing device with software “apps” to implement one or more methods as described herein or assist in implementing such procedures. In still another embodiment, computing system 52 may be implemented as or in association with an analog computing device, such as that disclosed, for example, in U.S. Pat. No. 6,751,501, which is incorporated herein by reference in its entirety. An example of a dated analog type computer or computing device is shown in FIG. 7. Such a device can generate various electronic waveforms, which can be selected for the type of control desired. In other words, devices such as that disclosed in U.S. Pat. No. 6,751,501 and FIG. 7 can be adapted for use or designed with contemporary electrical components for use with the disclosed embodiments herein.

In some embodiments, the computing system 52 may include a memory 57 for recording signals provided electronically from a detector for detecting a signal associated with pancreatic cancer in a body. Such a signal may derive from the detector 58 or from other detecting sources, as represented by dashed arrow 63 shown in FIG. 2. Dashed arrow 63 represents a signal associated with pancreatic cancer 60 in a human body. Computing system 52 can further include a processor 59 for reprogramming the signal 63 obtained from the memory 57 to generate a confounding treatment signal(s) 62 with respect to the pancreatic cancer 60.

The special detection system 58 can in some cases use modified LAB-view software (or similar software applications or “apps”) to interpret audio and visual information relating to the hunt for further cancer cell invasion, so such cells can be destroyed. Arrow 64 shown in FIG. 2 indicates the activity or identifying cancer in this manner. The special lights are shined on the above mentioned proximity organs and associated living biological elements seeking the sound of malignancy which occurs when certain lights are used to mildly heat-up the cancer so as to trigger vibration and quivering of the cellular-wall (i.e., plasma membrane) to make sounds. The living cells react to the assault of the abnormal heat and are heard by the audio detection system and then interpreted by the computer system. Normal cells make a different sound than malignant cells; it is more settled, uniform, and present in tone.

Cancer cells, on the other hand, are more in a state of high energy with more warmth and activity as they work through the mitosis activity related to the continuous reproduction of more cancer cells. Such activity consists of splitting the plasma membrane, organelles, and cytoplasm into two distinct cancerous daughter cells. The creation of a new cancer cell occurs continuously, but varies as to the type or kind of cell. Probably about 10 hours (e.g., this is an estimation) is the fastest a human cancer cell can produce 2 similar cells. In another 10 hours, for example, the pair of cancer cells can produce 4 new cancer cells, and so on.

Computer probes, camera monitors, telescopic vision inspection devices, and electrodes discussed herein can be inserted by laparoscopic entry techniques into the abdomen via trocar established entry areas. Once the cancer cells are located and mapped as to the extent of their distribution over the pancreas, close-organs, blood vessels, and nerve distribution, the treatment electrodes are applied to transmit the ultra-low-voltage encoded codes-of-death onto the pancreatic cancer cells and it's metastasises. Example probes 51 and 53 are shown in FIG. 2. Example probe 51 is associated with the signals represented by arrow 63, and probe 53 is associated with the audio-light-optic system 58 and arrow 64. It can be assumed that probes 51 or 53 or other probes may be also utilized in some embodiments to deliver the treatment codes 62 to the cancer 60.

In general, there are no digital signals in the human brain or any part of the nervous system or any muscle group, glandular systems, or vital organs. Every signal in every cell that makes up the electrical process of any living cell operates only in an “analog mode”. This means that the cancerous pancreatic tumor and any of its metastatic spread is also operating in an analog technical language.

A digital computer to process the pancreatic analog signal can be employed because the available analog systems are not as advanced in handling tiny complex cellular signals found in human cells, including large numbers of pancreatic cancer cells. Therefore, A/D converter 56 and D/A converter 54 are preferably used in handling the signals in reprogramming cell intrinsic electrical systems. This must be carefully planned when programming malignant diseased cells of the pancreas or any of the nearby organs such as metastatic cancers of the liver, spleen, stomach, and gall bladder. Reprogramming the associated arteries and veins that are encased in metastatic pancreatic cancer cells need to be treated with the “codes of death” to save the blood vessels by killing the overwhelming malignancy.

The species of pancreatic cancer are likely to be the same genetic type throughout all of the involved human structures, including glandular and digestive elements of the pancreas.

Laparoscopic Approach to Deliver Treatment Codes

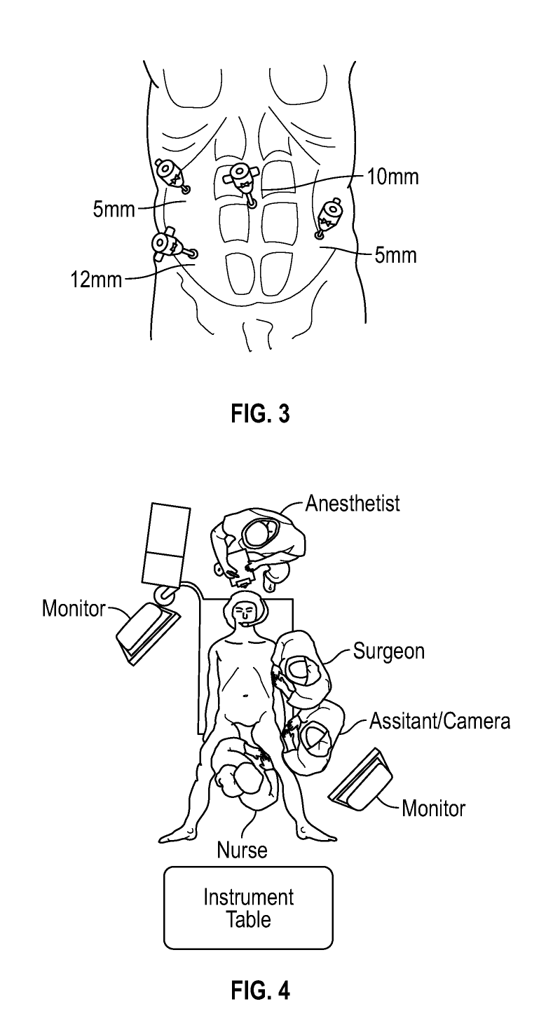

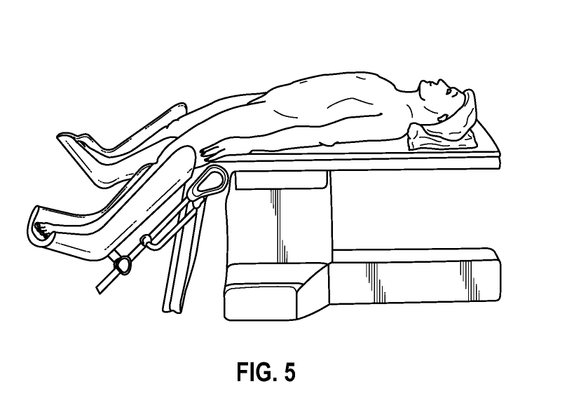

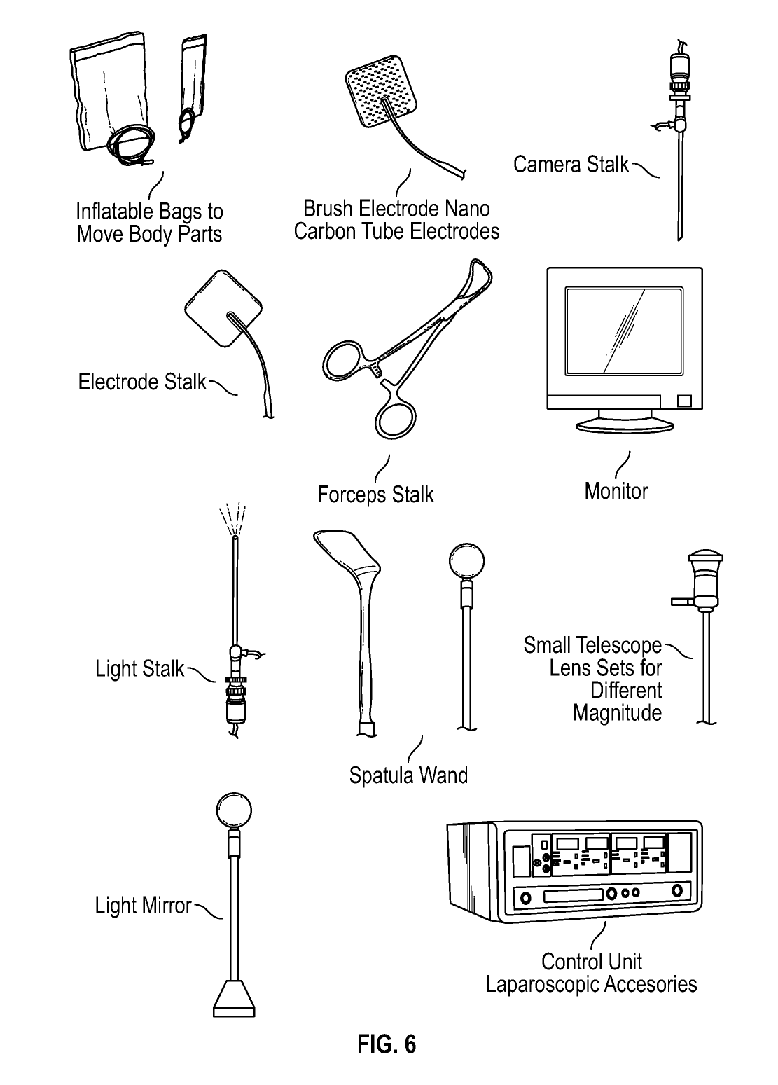

FIG. 3 illustrates a diagram of a human body, particularly a torso and abdominal area, with possible points of entry thereon for locating and destroying pancreatic cancer cells in accordance with an alternative embodiment. FIG. 4 illustrates a diagram of an example surgical operating room in which the disclosed procedure for locating and destroying pancreatic cancer cells may be carried out, in accordance with an alternative embodiment. FIG. 5 illustrates a side view of a patient in preparation for locating and destroying pancreatic cancer cells therein, in accordance with an alternative embodiment. FIG. 6 illustrates potential medical components and devices that can be utilized in accordance with the disclosed system and procedure for locating and destroying pancreatic cancer cells in accordance with an alternative embodiment.

Use of the abdominal laparoscopic approach as shown in FIGS. 3, 4, and 5 is likely safest to gain access to the pancreatic cancer cells by mapping their location and planning the delivery of the reprogrammed death-codes. The standard trocar entrances through the abdominal wall to supply monitor lights, cameras, and CO2 gas insufflation valve will be needed to slightly blot the abdominal wall to allow entrance and movement of the treatment devices and tools in the proper position. A camera will be inserted and lights that will be controlled by the monitoring assistant to provide the views and access for the medical staff and the operation of the cancer-code technology can come into play to get at the deadly pancreatic cancer cells. In addition, inflatable bumpers and spacers will need to be emplaced in the abdomen cavity to wedge-position the organs to allow the electrodes to attain the proper approach-angle to transmit codes-of-death into any cancer cells found. Adjustable multi-colored lights and different classification of bulbs to supply heat to trigger cancer-cell-wall vibrations that generate sounds that is different in malignant cells. The cellular vibrations and sounds to focus the electrodes and sensors to seek out the metastatic invasive cancer cells located on the pancreas and the surrounding organs, structures, and especially on the blood vessels to identify pools of malignancy and establish their exact location for destruction.

The bio-electronic signal processor 55 and locator system 58 can be designed to detect cancer cells that are anywhere within the abdomen that are associated with pancreatic cancer. All of the regional organs and structures that surround the pancreas are to be bio-electrically scanned for malignant cells. The hybrid analog-digital computerized system 52 can map the location of any cancers and store that data in the system 52, The electrodes, which are largely brush electrode constructed of many nano carbon electrode tubes that are connected to the recording and output leads of the technical system. The electrodes are identified as an imulus and are multi-purpose, being able to release chemicals and various ions from the hollow carbon filament that make up the operating tip of the system.

No surgery is utilized other than the use of the trocar to cut up to five 5 mm to 15 mm access-ports to allow entry for the bio-electronic analog/digital computer system plus instruments, probes, viewing telescope, and monitoring cameras among the necessary tools required to kill the living cancer cells. Potentially two trocar incisions of approximately 3 cm in length may be useful for access by manipulation instruments and the scientific instruments and devices to position the angle of approach as to the malignancies encountered in the region of the pancreatic cancer and associated organs.

The disclosed approach focuses on switching-off or damaging the cellular electrical system of the internal electrical communication system found in every cancer cell. Serious damage to the cellular electrical communication system will lead to death of the cancer cells, so damaged.

The bioelectronic system 50 can therefore program and re-program both the mapping software and the treatment cell codes. The unit can transmit and record cancer cell codes as well as modify the data associated with the technology.

Lights can be used with probes to detect and heat the cell walls and trigger sounds from the cancer that are different in tones and sound than normal healthy cells would sound-like. The light scan can be used at the completion of the treatment to confirm that all malignant cells are gone.

The Approach to Destroying the Pancreatic Cancer Cells

In some embodiments, the various instruments and sensors such as microphones, special lights, optical telescopes, etc., can be inserted at the proper entry points to: detect, inspect, and record cancer cells codes, and determine the position of the internal organs in the region of the pancreas so that the confounding treatment signals are transmitted directly into the pancreatic malignant cells, wherever they are located.

The application time of the confounding treatment signal 62, for example, can apply to the cancer 60 for approximately ten minutes to kill the malignant cells. The application of the treatment signal 62 may travel throughout a tumor for a certain distance and kill those cells that react to the confounding signal. Additional treatment sessions may be required to destroy a majority or all of the cancer cells.

If there are multiple pancreatic tumors, it may be necessary to employ the analog-digital bio-electronic system to confirm that the cancer internal operating codes are the same or different among any isolated tumors found in the abdomen. The Lab VIEW software, which is a product of National Labs, can be modified to record the internal cellular operating signals of the cancer. The embodiments disclosed herein focus on switching-off the internal communication and operating signals of the cancer cells. All of the cancer cells are linked to adjacent malignant cells by desmosomes, gap junctions, or tight junctions as the coupling between individual cells. Those connections allow the messages to travel between the cells. Within the cytoskeleton, there are microfilaments and intermediate filaments that carry the messages within the cell and between the adjoining cells of the tumor or metastatic deposits.

After recording the resident cellular operating codes, the reprogrammed lab-view software can compute the confounding signal necessary for destroying that particular family of cancer cells in the identified cancerized regions.

Embodiments can be equipped to wreck the electrical signals that operate the individual cancer cells. All of the cells in a tumor are related genetically and utilize the same set of codes to operate the cell and to communicate with adjacent cells. Once the abdominal area has been mapped as to the location of the cancer, the system is switched over to treatment mode. Treatment consists of reading and identifying the cancer signals and reprogramming such signals to shut down the signals and trigger necrosis, apoptosis, or karyorrhexis to accomplish programmed cell death of the cancer cells.

The cells are only operational electrically in the analog mode. The cancer cells do not react to digital electric signals. In fact, the entire body's electric system is analog and will respond only to that particular signaling format. The signals that influence the cancer cells are in the order of micro-volts (e.g., millionths of a volt) at pico-amps (e.g., trillionth of an amp). If digital signals were used at the power level of micro-volts at pico-amps, there would be no reaction in the cancer cells receiving the signal. Using digital waveforms at considerable higher power levels would only burn the cells. There would be no control of the cancer cell electrical system, nor would the signal speak to the nucleus. It must be formatted into flexible-analog delivery mode, which means the signal has the ability to vary itself as to amplitude and time. A musical orchestra is analog in its nature. All of the instruments are infinitely variable within the capability of the musical sound ranges it possesses.

An analog “sound” can be smeared or slid so as to sound slightly different. Digital sounds are on or off, ones or zeros. Humans and animals only operate and signal in an analog mode as a being. The very first simple example of living cells had no nucleus and was called prokaryote. The more advanced cell of which humans are composed of has a nucleus, which is analogous to a brain. This evolution into a cell with a nucleus and organelles enclosed within a membrane was more complex and classified as a eukaryote. Cells are the most primitive examples of actual life. About 100 trillion eukaryote cells make up a human body.

The hybrid system utilizes an analog/digital hybrid computerized system that can detect and map the location of any pancreatic cancer cells wherever they may be found within the abdominal cavity. Using Lab VIEW, for example, or other appropriate software, the treatment signals can be reprogrammed or adjusted to kill the pancreatic cancers found within the infected zone(s). Such system can be used repeatedly to seek live cancer cells and can repeat the treatment to destroy any remain living cancer cells. The hybrid computer system can shift program selections that can select various cell-death formats to be broadcast or transmit or be conducted into the pancreatic cells.

Based on the foregoing, it can be appreciated that a number of embodiments are disclosed herein. For example, in one embodiment a method can be implemented for treating pancreatic cancer. Such a method can include, for example, the steps of detecting a signal associated with pancreatic cancer in a body; recording the signal; and reprogramming the signal to generate a confounding treatment signal(s) with respect to a pancreatic cancer. In another embodiment, a step can be provided for aiming the confounding treatment signal at the pancreatic cancer in a body to shut down pancreatic cancer cells associated with the pancreatic cancer in the body. The least one confounding treatment signal generally comprises tiny encoded signals powered by ultra-low voltage approximated to a power level of a pancreatic cancer target comprising live pancreatic cancer cells.

In another embodiment, a step can be implemented for identifying the pancreatic cancer cells. Such a step can include, for example, seeking out via an audio-light-optic system, spreading pancreatic cancer cells that have moved onto nerves and blood vessels and the near-by organs which include at least: liver, stomach, gall bladder, spleen, and duodenum. Identifying the pancreatic cancer cells can further include scanning and locating aberrant cancer cell islands in the body using an audio-light-optic system. Identifying the pancreatic cancer cells can also include employing an audio-light-optic system to heat up and illuminate internal areas of the body working in concern with an audio system to record particular sounds associated with cancer.

In another embodiment, a step can be implemented for sorting noises associated with the body and sounds of non-malignant cells from the particular sounds associated with cancer. In yet another embodiment, a step can be implemented for generating a rendering of suspicious regions of active pancreatic cancer in the body after sorting the noises associated with the body and sounds of non-malignant cells from the particular sounds associated with cancer. In still another embodiment, a step can be provided for directing the confounding treatment signal toward the suspicious regions of active pancreatic cancer.

In another embodiment, a step can be provided for triggering, for example, necrosis, apoptosis, or karyorrhexis of pancreatic cancer cells associated with the pancreatic cancer by degrading a cellular electrical communication system of pancreatic cancer cells and tumors via the confounding treatment signal. In yet another embodiment, a step can be provided for transmitting the confounding treatment signal into the pancreatic cancer to invoke the triggering.

In another embodiment, a step can be implemented for entering the body via an abdomen with a laparoscope using a trocar to establish at least a three-entry incision of at least 5 mm and with at least two electrodes, at least one microphone, and in association with a closed-circuit televised monitor/audio system to record the view of a location of a tumor and sounds associated with the pancreatic cancer cells. In another embodiment, a step can be implemented for employing at least one Faraday enclosure in association with a plurality of lights with an audio recorder system associated with the audio-light-optic system either external or inside an abdomen to trigger the cellular sounds associated with the pancreatic cancer for use in the identifying the pancreatic cancer cells.

In another embodiment, a system can be implemented for treating cancer. Such a system can include a detector for detecting a signal associated with pancreatic cancer in a body; a memory for recording the signal provided electronically from the detector; and a processor for reprogramming the signal obtained from the memory to generate at least one confounding treatment signal with respect to a pancreatic cancer. The confounding treatment signal can be aimed electronically at the pancreatic cancer in a body to shut down pancreatic cancer cells associated with the pancreatic cancer in the body.

In general, the confounding treatment signal comprises tiny encoded signals powered by ultra-low voltage approximated to a power level of a pancreatic cancer target comprising live pancreatic cancer cells. In yet another embodiment, an audio-light-optic system can be provided for identifying the pancreatic cancer cells. The audio-light-optic system can be employed to seek out in the body spreading pancreatic cancer cells that have moved onto nerves and blood vessels and the near-by organs which include at least: liver, stomach, gall-bladder, spleen, and duodenum of the body. The audio-light-optic system can scan and locate aberrant cancer cell islands in the body.

In another embodiment, the audio-light-optic system can be employed to heat up and illuminate internal areas of the body working in concert with an audio system to record particular sounds associated with cancer. In yet another embodiment, the audio-light-optic system can assist in sorting noises associated with the body and sounds of non-malignant cells from the particular sounds associated with cancer. The audio-light-optic system can also assist in generating a rendering of suspicious regions of active pancreatic cancer in the body after sorting the noises associated with the body and sounds of non-malignant cells from the particular sounds associated with cancer. Such a rendering can be displayed on a display such as, for example, the monitors shown in FIG. 4 and FIG. 6.

In general, the confounding treatment signal can be directed toward the suspicious regions of active pancreatic cancer. The confounding treatment signal is capable of triggering at least one of necrosis, apoptosis, or karyorrhexis of pancreatic cancer cells associated with the pancreatic cancer by degrading a cellular electrical communication system of pancreatic cancer cells and tumors via the confounding treatment signal. The confounding treatment signal can be transmitted into the pancreatic cancer to invoke the triggering.

In another embodiment, a laparoscope can be employed, which enters the body via an abdomen using a trocar to establish at least a three-entry incision of at least 5 mm and with at least two electrodes, at least one microphone, and in association with a closed-circuit televised monitor/audio system to record a view of a location of a tumor and sounds associated with the pancreatic cancer cells. In yet another embodiment, a Faraday enclosure can be implemented in association with a plurality of lights with an audio recorder system associated with the audio-light-optic system either external or inside an abdomen to trigger the cellular sounds associated with the pancreatic cancer for use in the identifying the pancreatic cancer cells.

It will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

What is claimed is:

1. A method for treating pancreatic cancer, said method comprising:
detecting and recording a signal associated with pancreatic cancer in a body; and
reprogramming said signal to generate at least one confounding treatment signal with respect to a pancreatic cancer.

2. The method of claim 1 further comprising aiming at least one confounding treatment signal at said pancreatic cancer in a body to shut down pancreatic cancer cells associated with said pancreatic cancer in said body.

3. The method of claim 1 wherein said at least one confounding treatment signal comprises tiny encoded signals powered by ultra-low voltage approximated to a power level of a pancreatic cancer target comprising live pancreatic cancer cells.

4. The method of claim 2 further comprising identifying said pancreatic cancer cells.

5. The method of claim 4 wherein identifying said pancreatic cancer cells further comprises seeking out via an audio-light-optic system, spreading pancreatic cancer cells that have moved onto nerves and blood vessels and the near-by organs which include at least: liver, stomach, gall-bladder, spleen, and duodenum.

6. The method of claim 4 wherein identifying said pancreatic cancer cells further comprises scanning and locating aberrant cancer cell islands in said body using an audio-light-optic system.

7. The method of claim 4 wherein identifying said pancreatic cancer cells further comprises employing an audio-light-optic system to heat up and illuminate internal areas of said body working in concern with an audio system to record particular sounds associated with cancer.

8. The method of claim 1 further comprising sorting noises associated with said body and sounds of non-malignant cells from said particular sounds associated with cancer.

9. The method of claim 8 further comprising generating a rendering of suspicious regions of active pancreatic cancer in said body after sorting said noises associated with said body and sounds of non-malignant cells from said particular sounds associated with cancer.

10. The method of claim 9 further comprising directing said at least one confounding treatment signal toward said suspicious regions of active pancreatic cancer.

11. The method of claim 1 further comprising triggering at least one of necrosis, apoptosis, or karyorrhexis of pancreatic cancer cells associated with said pancreatic cancer by degrading a cellular electrical communication system of pancreatic cancer cells and tumors via said at least one confounding treatment signal.

12. The method of claim 11 further comprising transmitting said at least one confounding treatment signal into said pancreatic cancer to invoke said triggering.

13. The method of claim 2 further comprising entering said body via an abdomen with a laparoscope using a trocar to establish at least a three-entry incision of at least 5 mm and with at least two electrodes, at least one microphone, and in association with a closed-circuit televised monitor/audio system to record the view of a location of a tumor and sounds associated with said pancreatic cancer cells.

14. The method of claim 4 further comprising employing at least one Faraday enclosure in association with a plurality of lights with an audio recorder system associated with said audio-light-optic system either external or inside an abdomen to trigger the cellular sounds associated with said pancreatic cancer for use in said identifying said pancreatic cancer cells.

15. A system for treating pancreatic cancer, said system comprising:
a detector for detecting a signal associated with pancreatic cancer in a body;
a memory for recording said signal provided electronically from said detector; and
a processor for reprogramming said signal obtained from said memory to generate at least one confounding treatment signal with respect to a pancreatic cancer.

16. The system of claim 15 wherein at least one confounding treatment signal is aimed electronically at said pancreatic cancer in a body to shut down pancreatic cancer cells associated with said pancreatic cancer in said body.

17. The system of claim 15 wherein said at least one confounding treatment signal comprises tiny encoded signals powered by ultra-low voltage approximated to a power level of a pancreatic cancer target comprising live pancreatic cancer cells.

18. The system of claim 15 wherein a detector further comprises an audio-light-optic system for identifying said pancreatic cancer cells.

19. The method of claim 18 wherein said audio-light-optic system for identifying said pancreatic cancer cells seeks out in said body spreading pancreatic cancer cells that have moved onto nerves and blood vessels and the near-by organs which include at least: liver, stomach, gall-bladder, spleen, and duodenum of said body.

20. The system of claim 18 wherein said an audio-light-optic system for identifying said pancreatic cancer cells scans and locates aberrant cancer cell islands in said body using an audio-light-optic system and wherein said audio-light-optic system heats up and illuminates internal areas of said body working in concert with an audio system to record particular sounds associated with cancer.
